(19) United States
(12) Patent Application Publication
(10) Pub. No.: US 2010/0056547 A1
(43) Pub. Date: Mar. 4, 2010

(54) OPHTHALMIC COMPOSITIONS COMPRISING dDC

(75) Inventors: Patrick M. Hughes, Aliso Viejo, CA (US); Rhett M. Schiffman, Laguna Beach, CA (US); Harold G. Jensen, Lake Forest, CA (US); Joan-En Chang-Lin, Tustin, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/909,479

(22) PCT Filed: Mar. 21, 2006
(86) PCT No.: PCT/US2006/010176

§ 371 Date: Nov. 16, 2009

Related U.S. Application Data
(60) Provisional application No. 60/665,170, filed on Mar. 25, 2005.
(60) Provisional application No. 60/728,020, filed on Oct. 17, 2005.

(51) Int. Cl. A61K 31/495; A61P 27/02

(52) U.S. Cl. 514/256

(57) ABSTRACT

A composition comprising dDC and a polymer, wherein the composition is an aqueous liquid with a viscosity which increases upon contact with a surface of an eye is disclosed herein.

An aqueous composition comprising a therapeutically effective concentration of dDC, wherein the concentration of dDC is less than 1% is also disclosed.

An eye drop comprising a therapeutically effective amount of dDC, wherein the amount of dDC is less than 300 μg is also disclosed.

A method comprising administering an effective amount of dDC topically to an eye of a person suffering from viral conjunctivitis a viral infection, wherein less than 300 μL of dDC is administered to said eye is also disclosed.

A kit comprising a composition and a dispenser, wherein said dispenser dispenses a drop comprising a therapeutically effective amount of dDC, wherein the amount of dDC is less than 300 μg is also disclosed.

### CROSS-REFERENCE TO RELATED APPLICATIONS

This is a national stage application under 35 U.S.C. § 371 of PCT application PCT/US2006/010176, filed Mar. 21, 2006, which claims the benefit of U.S. Provisional Application Ser. No. 60/665,170, filed Mar. 25, 2005, and U.S. Provisional Application Ser. No. 60/728,020, filed Oct. 17, 2005, and each of which is incorporated by reference in their entirety herein.

### DESCRIPTION OF RELATED ART

Viral conjunctivitis, known commonly as pink eye, is a common highly contagious disease. Topical ophthalmic administration of cidofavir has been shown to be effective in treating viral conjunctivitis by Gordon et. al. (Cornea 15(5): 546, 1996).

2′,3′-Dideoxycytidine (dDC), also known as zalcitabine, is an antiviral drug which is used to treatment of HIV patients having the structure shown below.

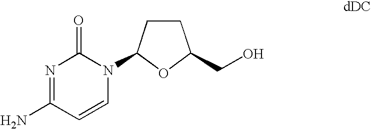

Kaneko et. al. (J. Jpn. Ophthalm. Soc. 104: 786-791 (2000)) stated “[f]uture research on development of HPMPC and dDC as eye drops for AdV conjunctivitis are promising.” In a later related study, Kaneko et. al. (J. Jpn. Ophthalmol. Soc. 107:196-201 (2003)) found that NMSO₃, HPMPC, and dDC inhibited adenoviral strains, but that NMSO₃ had the lowest EC50 values. Keneko further pointed out that there may be problems with adverse reactions related to the cytotocity of nucleic acid derivatives such as HPMPC and dDC.

WO2004/087203 discloses “methods and composition of an immunostimulatory nucleic acid in oil-in-water emulsions for topical delivery.” It further mentions that “[t[he immunostimulatory nucleic acid may be administered to the skin or to the mucosa. Mucosal administration include oral, ocular, nasal, vaginal, rectal and the like” and that “[i]n some embodiments the anti-viral agent is selected from the group consisting of Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine ; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine ; Zidovudine; and Zinviroxime.”

### BRIEF DESCRIPTION OF THE DRAWING FIGURES

FIG. 1 is a plot depicting the positive cultures per total for several antiviral compositions disclosed herein.

FIG. 2 is a plot depicting the mean duration of shedding for several antiviral compositions disclosed herein.

FIG. 3 is a plot depicting the mean daily ocular titers for several antiviral compositions disclosed herein.

### DESCRIPTION OF THE INVENTION

A composition comprising dDC and a polymer, wherein the composition is an aqueous liquid with a viscosity which increases upon contact with a surface of an eye is disclosed herein.

While not intending to limit the scope of the invention in any way, one method of increasing the viscosity of the composition upon contact with the eye is to administer a composition to the eye which has a viscosity that is pH dependent. Thus, the pH of the composition would be different than that of the eye, but still tolerable to the eye, and the composition would have a higher viscosity at the pH of the eye than at the pH of the composition before administration.

In one embodiment the viscosity of the composition is pH dependent. In another embodiment the viscosity of the composition is less than 15,000 cps at pH 4. In another embodiment the viscosity of the composition is greater than 50,000 cps at pH 7.

In another embodiment, the viscosity of the composition is 12,000 cps or less.

In another embodiment the viscosity of the composition becomes 70,000 or greater upon contact with the surface of the eye.

In another embodiment the viscosity of the composition increases by a factor of 5 or more upon contact with the surface of the eye.

The term “polymer” has the meaning commonly understood in the art. While not intending to limit the scope of the invention in any way, the polymer may often be useful for increasing the viscosity of the composition upon contact with the surface of the eye. For example, ionic polymers are sometimes useful for modulating viscosity of a liquid composition. While not intending to be bound in any way by theory, or to limit the scope of the invention in any way, the viscosity of ionic polymers may be dependent upon the pH of a composition. Thus, ionic polymers are useful for preparing a composition whose viscosity is pH dependent. Ionic polymers can be, among other things, anionic, cationic, or zwitterionic. One embodiment, comprises an anionic polymer. Another embodiment comprises a polycarboxylic acid such as a polymer of acrylic acid. In another embodiment, the polymer is a polyamine.

Other compositions comprise both a polyionic polymer and one or more additional polymers. In one embodiment, both a polyanionic polymer and one or more additional polymers is present. Another embodiment comprises a polymer of acrylic acid and a cellulose ether. Another embodiment comprises a carbomer and a cellulosic polymer.

Another embodiment comprises 0.8% dDC, 0.5% Methocel A4M premium, 0.3% Carbapol 934P, 2.2% glycerine, wherein the composition has a pH of 4.

Unless otherwise indicated, reference to % in the specification or claims herein is intended to mean % (weight/volume).

An aqueous composition comprising a therapeutically effective concentration of dDC, wherein the concentration of dDC is less than 1% is also disclosed herein.

In one embodiment the concentration of dDC is from 0.01% to 0.99%. In another embodiment the concentration of dDC is from 0.2% to 0.8%. In one embodiment the concentration of dDC is at least 0.4%. In another embodiment the concentration of dDC is no greater than about 0.8%. In another embodiment the concentration of dDC is about 0.8%.

An eye drop comprising a therapeutically effective amount of dDC, wherein the amount of dDC is less than 300 μg is also disclosed herein.

The amount of dDC in an eye drop is determined by both the concentration of the eye drop solution and the size of the eye drop. In one embodiment the size of the eye drop is from 30 μL to 35 μL. In another embodiment, the size of the eye drop is from 5 to 20 μL. In another embodiment, the size of the eye drop is from 5 to 10 μL.

In one embodiment the concentration of dDC in the eye drop is from 0.01% to 3%. In another embodiment the concentration of dDC in the eye drop is from 1% to 2%. In another embodiment the concentration of dDC in the eye drop is from 0.01% to 0.99%. In another embodiment the concentration of dDC in the eye drop is from 0.2% to 0.8%. In one embodiment the concentration of dDC in the eye drop is at least 0.4%. In another embodiment the concentration of dDC in the eye drop is no greater than about 0.8%. In another embodiment the concentration of dDC in the eye drop is about 0.8%.

In one embodiment the amount of dDC in the eye drop is 280 μg or less. In another embodiment the amount of dDC in the eye drop is from 150 μg to 299 μg.

A method comprising administering an effective amount of dDC topically to an eye of a person suffering from a viral infection, wherein less than 300 μg of dDC is administered to said eye is also disclosed herein. In other words, the eye receives less than 300 μg in each individual treatment. This method may be practiced on both eyes simultaneously. In other words, a person may receive a treatment or dose of less than 300 μg of dDC to one eye, and another treatment or dose of less than 300 μg of dDC to the second eye, at or around the same time.

In one embodiment, a dose of dDC having less than 300 μg of dDC, is administered no more than 6 times a day. These six doses may occur in any combination to one or both eyes. In other words, in one embodiment a dose having no more than 300 μg of dDC is administered to both eyes of the person no more than 3 times a day. In another embodiment, a dose having no more than 300 μg of dDC is administered to only one eye no more than 6 times a day. In other embodiments, both eyes receive 1 or more dose of no more than 300 μg of dDC per day, but the total number of doses does not exceed six. For example, one eye may receive 2 doses and the other eye receive 4 doses; one eye may receive 1 dose and the other eye may receive 5 doses; one eye may receive 2 doses and the other eye may receive 3 doses; etc.

A kit comprising a composition and a dispenser, wherein said dispenser dispenses a drop comprising a therapeutically effective amount of dDC, wherein the amount of dDC is less than 300 μg is also disclosed herein.

A composition comprising from 0% to 3% dDC and a penetration enhancer, wherein said composition is ophthalmically acceptable is also disclosed herein.

A penetration enhancer is a compound or a mixture of compounds which enhances penetration of a molecule through tissue. Penetration enhancers may enhance penetration of the compound by improving permeation either through the cell itself (trancellular), between the cells (paracellular), or both. While not intending to limit the scope of the invention in any way, examples of trancellular penetration enhancers include surfactants, bile salts, fatty acids, cyclodextrins, vitamin E tocopherol, or combinations thereof. Examples of paracellular penetration enhancers include preservatives, chelating agents, and liposomes or vesicles, or combinations thereof.

Examples of useful surfactants include, but are not limited to

Spans 20, 40 and 1%;

Tweens 20, 40 and 81;

Aptet 100;

G 1045;

Brij 35, 58, 78, 98;

Myrj 52 and 53; and

BL-9.

Examples of useful bile acids include, but are not limited to

deoxycholic acid,

taurocholic acid,

taurodeoxycholic acid,

ursodeoxycholic acid, and

tauroursodeoxycholic acid.

Examples of useful fatty acids include, but are not limited to capric acid and those mentioned elsewhere herein.

Examples of useful preservatives include, but are not limited to

Benzalkonium chloride (BAK), chlorhexidine digluconate, benzyl alcohol, chlorobutanol, 2-phenylethanol, paraben, and propyl paraben Examples of useful chelating agents include, but are not limited to, ethylenediaminetetraacetic acid (EDTA).

Other useful penetration enhancers include azone, hexamethylene lauramide, hexamethylene octanamide, decylmethylsulfoxide, and saponin.

One embodiment comprises from 0 to 3% dDC and Span 20%, Span 40%, or Span 1%.

Another embodiment comprises from 0 to 3% dDC and Tween 20, Tween 40, or Tween 81.

Another embodiment comprises from 0 to 3% dDC and Aptet 100. Another embodiment comprises from 0 to 3% dDC and G 1045.

Another embodiment comprises from 0 to 3% dDC and Brij 35, Brij 58, Brij 78, or Brij 98.

Another embodiment comprises from 0 to 3% dDC and Myrj 52 or Myrj 53.

Another embodiment comprises from 0 to 3% dDC and BL-9.

Another embodiment comprises from 0 to 3% dDC and deoxycholic acid.

Another embodiment comprises from 0 to 3% dDC and taurocholic acid.

Another embodiment comprises from 0 to 3% dDC and taurodeoxycholic acid.

Another embodiment comprises from 0 to 3% dDC and ursodeoxycholic acid.

Another embodiment comprises from 0 to 3% dDC and tauroursodeoxycholic acid.

Another embodiment comprises from 0 to 3% dDC and capric acid.

Another embodiment comprises from 0 to 3% dDC and benzalkonium chloride (BAK).

Another embodiment comprises from 0 to 3% dDC and chlorhexidine digluconate.

Another embodiment comprises from 0 to 3% dDC and benzyl alcohol. Another embodiment comprises from 0 to 3% dDC and chlorobutanol.

Another embodiment comprises from 0 to 3% dDC and 2-phenylethanol.

Another embodiment comprises from 0 to 3% dDC and paraben.

Another embodiment comprises from 0 to 3% dDC and propyl paraben. Another embodiment comprises from 0 to 3% dDC and ethylenediaminetetraacetic acid (EDTA).

Another embodiment comprises from 0 to 3% dDC and azone. Another embodiment comprises from 0 to 3% dDC and hexamethylene lauramide.

Another embodiment comprises from 0 to 3% dDC and hexamethylene octanamide.

Another embodiment comprises from 0 to 3% dDC and decylmethylsulfoxide.

Another embodiment comprises from 0 to 3% dDC and saponin.

A composition comprising dDC and a fluorocarbon is also disclosed herein. Another composition comprises from 0.01% to 3% dDC and a fluorocarbon.

While not intending to limit the scope of the invention in any way, certain compositions are fluorocarbon based rather than water based. In other words, a fluorocarbon may be the primary solvent of the composition.

While not intending to limit the scope of the invention, perfluorodecalin is a useful fluorocarbon.

Another embodiment comprises an organic acid salt of dDC. The organic acid is any organic compound having a pKa less than about 4.47. While not intending to limit the scope of the invention in any way, suitable organic acids include:

Carboxylic acids including

fatty acids which are linear carboxylic acids having an even number of carbon atoms;

Saturated fatty acids have no C═C moieties and include, but are not limited to, myristic acid, palmitic acid, stearic acid, arachidic acid, and behenic acid;

unsaturated fatty acids, including, but not limited to, the following:

monounsaturated fatty acids, which have one C═C group such as palmitoleic acid and oleic acid;

diunsaturated fatty acids, which have two C═C groups, such as linoleic acid;

triiunsaturated fatty acids, which have three C═C groups, such as α-linolenic acid and γ-linolenic acid;

tetraunsaturated fatty acids, which have four C═C groups, such as arachidonic acid; and

pentaunsaturated fatty acids, which have five C═C groups, such as eicosapentaenoic acid;

Sulfur acids including sulfonic acids; and

Phosphorous acids including phosphonic acids.

While not intending to limit the scope of the invention in any way, one embodiment comprises salts of organic acids having from 4 to 24 carbon atoms. Another embodiment comprises salts of carboxylic acids. Another embodiment comprises salts of carboxylic acids having from 4 to 24 carbon atoms. Another embodiment comprises lipophilic salts with log P values in the 2 to 4 range. While not intending to limit the scope of the invention in any way, palmoate, stearate, palmitate, laurate, octanoate, mysterate, caproate, butyrate, lauryl sulfate, decanoate, benzoate, procaine salts, are examples of useful dDC salts.

Another embodiment comprises a prodrug of dDC having a log P of from 2 to 4.

Another embodiment comprises

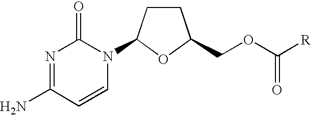

or a pharmaceutically acceptable salt thereof;
wherein R is hydrocarbyl or heterohydrocarbyl having from 3 to 24 carbon atoms.

Hydrocarbyl is a moiety consisting of only carbon and hydrogen, and includes, but is not limited to alkyl, alkenyl, alkynyl, and the like, and in some cases aryl, and combinations thereof.

Alkyl is hydrocarbyl having no double or triple bonds including:

linear alkyl such as methyl, ethyl, propyl, n-butyl, n-pentyl, n-hexyl, and the like;

branched alkyl such as isopropyl, branched butyl isomers (i.e. sec-butyl, tert-butyl, etc), branched pentyl isomers (i.e. isopentyl, etc), branched hexyl isomers, and higher branched alkyl fragments;

cycloalkyl such as cyclopropyl, cyclobutyl, cyclopentyl, cyclohexyl, cycloheptyl, etc.; and

alkyl fragments consisting of both cyclic and noncyclic components, whether linear or branched, which may be attached to the remainder of the molecule at any available position including terminal, internal, or ring carbon atoms.

Alkenyl is hydrocarbyl having one or more double bonds including linear alkenyl, branched alkenyl, cyclic alkenyl, and combinations thereof in analogy to alkyl.

Alkynyl is hydrocarbyl having one or more triple bonds including linear alkynyl, branched alkynyl, cyclic alkynyl and combinations thereof in analogy to alkyl.

Aryl is an unsubstituted or substituted aromatic ring or ring system such as phenyl, naphthyl, biphenyl, and the like. Aryl may or may not be hydrocarbyl, depending upon whether it has substituents with heteroatoms.

Arylalkyl is alkyl which is substituted with aryl. In other words alkyl connects aryl to the remaining part of the molecule. Examples are —CH₂-Phenyl, —CH₂—CH₂-Phenyl, and the like. Arylalkyl may or may not be hydrocarbyl, depending upon whether it has substituents with heteroatoms.

Unconjugated dienes or polyenes have one or more double bonds which are not conjugated. They may be linear, branched, or cyclic, or a combination thereof.

Combinations of the above are also possible.

Heterohydrocarbyl is a moiety comprising heteroalkyl, heteroaryl, or heteroatom substituted aryl, or a combination thereof, either alone or in combination with hydrocarbyl and/or aryl. The following moieties are typical examples.

Heteroalkyl is alkyl having one or more carbon atoms substituted with O or S atoms, provided that no carbon has more than 1 covalent bond to O or S, i.e. there are no C═O, —O—CH₂—O—, —S—CH₂—S—, etc.; and that O and S are only bonded to carbon, i.e. there are no OH, SH, SO₃H, etc.

Heteroatom substituted aryl comprises one or more substituents on the aryl ring or ring system, said substituents comprising one or more atoms which are not H or C.

Heteroatom substituted arylalkyl comprises one or more substituents on the aryl ring or ring system, said substituents comprising one or more atoms which are not H or C.

Heteroaryl is aryl having one or more N, O, or S atoms in the ring, i.e. a ring carbon is substituted by N, O, or S. While not intending to be limiting, examples of heteroaryl include unsubstituted or substituted thienyl, pyridinyl, furyl, benzothienyl, benzofuryl, imidizololyl, indolyl, and the like.

Heteroarylalkyl is alkyl which is substituted with heteroaryl. In other words alkyl connects heteroaryl to the remaining part of the molecule. Examples are —CH₂-thienyl, —CH₂CH₂-benzothienyl, and the like.

Heteroarylheteroalkyl is heteroalkyl which is substituted with heteroaryl. In other words heteroalkyl connects heteroaryl to the remaining part of the molecule. Examples are —CH₂O-thienyl, —CH₂S-benzothienyl, and the like.

Arylheteroalkyl is heteroalkyl which is substituted with aryl. In other words heteroalkyl connects aryl to the remaining part of the molecule. Examples are —CH₂O-phenyl, —CH₂S-naphthyl, and the like.

The substituents of aryl or heteroaryl may have up to 12 non-hydrogen atoms each and as many hydrogen atoms as necessary. Thus, while not intending to limit the scope of the invention in any way, the substituents may be:

hydrocarbyl, such as alkyl, alkenyl, alkynyl, and the like, and combinations thereof;

hydrocarbyloxy, meaning O-hydrocarbyl such as OCH₃, OCH₂CH₃, O-cyclohexyl, etc, up to 11 carbon atoms;

hydroxyhydrocarbyl, meaning hydrocarbyl-OH such as CH₂OH, C(CH₃)₂OH, etc, up to 11 carbon atoms;

nitrogen substituents such as NO₂, CN, and the like, including

amino, such as NH₂, NH(CH₂CH₃OH), NHCH₃, and the like up to 11 carbon atoms;

carbonyl substituents, such as CO₂H, ester, amide, and the like;

halogen, such as chloro, fluoro, bromo, and the like

fluorocarbyl, such as CF₃, CF₂CF₃, etc.;

phosphorous substituents, such as PO₃²⁻, and the like;

sulfur substituents, including S-hydrocarbyl, SH, SO₃H, SO₂-hydrocarbyl, SO₃-hydrocarbyl, and the like.

In certain embodiments, the number of non-hydrogen atoms is 6 or less in a substituent. In other embodiments, the number of non-hydrogen atoms is 3 or less in a substituent. In other embodiments, the number of non-hydrogen atoms on a substituent is 1.

In certain embodiments, the substituents contain only hydrogen, carbon, oxygen, halogen, nitrogen, and sulfur. In other embodiments, the substituents contain only hydrogen, carbon, oxygen, and halogen.

While not intending to limit the scope of the invention in any way, an esterification procedure can used to synthesize the dDC esters. Acylation of dDC can be achieved by suspending the compound in a 1:1 solution of N,N-dimethyl formamide (DMF): pyridine and a 5-fold excess of the appropriate acid anhydride at room temperature. The reaction will be allowed to proceed for 2-3 days at room temperature until completion as ascertained by thin layer chromatographic (TLC) analysis. The pyridine and DMF is removed in vacuo and the product precipitated from the acid anhydride. The precipitate will be purified by silica gel chromatography and recrystallized from a benzene-methanol mixture to yield the purified product.

The compounds, methods, medicaments, and compositions disclosed herein are useful for the treatment of viral infections affecting the ocular surface or other parts of the eye.

Also contemplated herein is a kit comprising a dispenser and a label, said dispenser containing a composition disclosed herein, and said dispenser being capable of providing drops of said composition suitable for topical administration to an eye. Said kit may also contain a label indicating administration of said drops of said composition to an eye of a mammal.

Diseases or conditions which may be treated include viral conjunctivitis, viral keratitis, viral trabeculitis, viral iritis, viral scleritis, viral blethritis, viral vitritis, and other viral uveitides.

While not intending to limit the scope of the invention in any way, examples of viruses affecting the eye include
adenovirus (all serotypes);
Herpes virus including, but not limited to simplex type 1 & 2, varicella zoster, Epstein Barr, cytomegalovirus, and human herpes virus 6,7, and 8, and the like;
picomaviruses including, but not limited to enterovirus, Coxsackie, and the like;
poxvirus molluscum contagiosum, vaccinia).
paramyxovirus including, but not limited to measles, mumps, Newcastile, and the like; West Nile Virus; and
rubella virus and the like.

In one embodiment, the compositions are used to treat viral conjunctivitis or viral keratitis.

One embodiment is use of a compound in the manufacture of a medicament for the treatment of a viral infection affecting the eye, wherein said compound is dDC, and said medicament is an aqueous liquid with a polymer having a viscosity which increases upon contact with a surface of an eye.

Another embodiment is use of a compound in the manufacture of a medicament for the treatment of a viral infection affecting the eye, wherein said compound is dDC, and said medicament is an aqueous liquid with a polymer having a viscosity which increases upon contact with a surface of an eye, wherein the viscosity of said medicament is pH dependent.

Another embodiment is use of a compound in the manufacture of a medicament for the treatment of a viral infection affecting the eye, wherein said compound is dDC, and said medicament is an aqueous liquid with a polymer having a viscosity which increases upon contact with a surface of an eye, wherein the polymer is an anionic polymer.

Another embodiment is use of a compound in the manufacture of a medicament for the treatment of a viral infection affecting the eye, wherein the medicament comprises 0.8% dDC, 0.5% Methocel A4M premium, 0.3% Carbapol 934P, 2.2% glycerine, and has a pH of 4.

Another embodiment is use of a compound in the manufacture of a medicament for the treatment of a viral infection affecting the eye, wherein said medicament is an aqueous composition, and said compound is dDC having a concentration less than 1%.

Another embodiment is use of a compound in the manufacture of a medicament for the treatment of a viral infection affecting the eye, wherein said medicament is an aqueous composition, and said compound is dDC having a concentration of at least 0.4%.

Another embodiment is use of a compound in the manufacture of a medicament for the treatment of a viral infection affecting the eye, wherein said medicament is an aqueous composition, and said compound is dDC having a concentration of about 0.8%.

One embodiment is a kit comprising a composition and a dispenser, wherein the composition is an aqueous liquid comprising dDC with a viscosity which increases upon contact with a surface of an eye.

Another embodiment is a kit comprising a composition and a dispenser, wherein the composition is an aqueous liquid comprising dDC with a viscosity which increases upon contact with a surface of an eye, wherein the viscosity of the composition is pH dependent.

Another embodiment is a kit comprising a composition and a dispenser, said composition comprising dDC and a polymer, wherein the composition is an aqueous liquid with a viscosity which increases upon contact with a surface of an eye, wherein the viscosity of the composition is pH dependent, and wherein the viscosity of the composition is less than 15,000 cps at pH 4.

Another embodiment is a kit comprising a composition and a dispenser, said composition comprising dDC and a polymer, wherein the composition is an aqueous liquid with a viscosity which increases upon contact with a surface of an eye, wherein the viscosity of the composition is pH dependent, and wherein the viscosity of the composition is greater than 50,000 cps at pH 7.

Another embodiment is a kit comprising a composition and a dispenser, said composition comprising dDC and a polymer, wherein the composition is an aqueous liquid with a viscosity which increases upon contact with a surface of an eye, and wherein the viscosity of the composition is 12,000 cps or less.

Another embodiment is a kit comprising a composition and a dispenser, said composition comprising dDC and a polymer, wherein the composition is an aqueous liquid with a viscosity which increases upon contact with a surface of an eye, and wherein the viscosity of the composition is 12,000 cps or less, and wherein the viscosity of the composition becomes 70,000 or greater upon contact with the surface of the eye.

Another embodiment is a kit comprising a composition and a dispenser, said composition comprising dDC and a polymer, wherein the composition is an aqueous liquid with a viscosity which increases upon contact with a surface of an eye, and wherein the viscosity of said composition increases by a factor of 5 or more upon contact with the surface of the eye.

Another embodiment is a kit comprising a composition and a dispenser, said composition comprising dDC and a polymer, wherein the composition is an aqueous liquid with a viscosity which increases upon contact with a surface of an eye, and wherein the polymer is an anionic polymer.

Another embodiment is a kit comprising a composition and a dispenser, said composition comprising dDC and a polymer, wherein the composition is an aqueous liquid with a viscosity which increases upon contact with a surface of an eye, and wherein the polymer is an anionic polymer comprising a polymer of acrylic acid and a cellulose ether.

Another embodiment is a kit comprising a composition and a dispenser, said composition comprising dDC, a carbomer, and a cellulosic polymer.

Another embodiment is a kit comprising a composition and a dispenser, said composition comprising 0.8% dDC, 0.5% Methocel A4M premium, 0.3% Carbapol 934P, 2.2% glycerine, wherein the composition has a pH of 4.

Another embodiment is a kit comprising an aqueous composition and a dispenser, said comprising a therapeutically effective concentration of dDC, wherein the concentration of dDC is less than 1%.

Another embodiment is a kit comprising an aqueous composition and a dispenser, said composition comprising a therapeutically effective concentration of dDC, wherein the concentration of dDC is at least 0.4%.

Another embodiment is a kit comprising an aqueous composition and a dispenser, said comprising a therapeutically effective concentration of dDC, wherein the concentration of dDC is no greater than about 0.8%.

Another embodiment is a kit comprising an aqueous composition and a dispenser, said comprising a therapeutically effective concentration of dDC, wherein the concentration of dDC is about 0.8%.

Another embodiment is use of dDC in the manufacture of a medicament for the treatment of viral infection, wherein said viral infection affects an eye, and wherein the concentration of dDC is less than 1%.

Another embodiment is use of dDC in the manufacture of a medicament for the treatment of viral infection, wherein said viral infection affects an eye, and wherein the concentration of dDC is less than 1%, wherein said viral infection is a disease selected from the group consisting of viral conjunctivitis, viral keratitis, viral trabeculitis, viral iritis, viral scleritis, viral blethritis, viral vitritis, viral uveitides, and combinations thereof.

Another embodiment is use of dDC in the manufacture of a medicament for the treatment of viral infection, wherein said viral infection affects an eye, and wherein the concentration of dDC is less than 1%, wherein said viral infection is viral conjunctivitis or viral keratitis

One embodiment is a composition comprising dDC and a polymer, wherein the composition is an aqueous liquid with a viscosity which increases upon contact with a surface of an eye.

Another embodiment is a composition comprising dDC and a polymer, wherein the composition is an aqueous liquid with a viscosity which increases upon contact with a surface of an eye, wherein the viscosity of the composition is pH dependent.

Another embodiment is a composition comprising dDC and a polymer, wherein the composition is an aqueous liquid with a viscosity which increases upon contact with a surface of an eye, wherein the viscosity of the composition is pH dependent, and wherein the viscosity of the composition is less than 15,000 cps at pH 4.

Another embodiment is a composition comprising dDC and a polymer, wherein the composition is an aqueous liquid with a viscosity which increases upon contact with a surface of an eye, wherein the viscosity of the composition is pH dependent, and wherein the viscosity of the composition is greater than 50,000 cps at pH 7.

Another embodiment is a composition comprising dDC and a polymer, wherein the composition is an aqueous liquid with a viscosity which increases upon contact with a surface of an eye, and wherein the viscosity of the composition is 12,000 cps or less.

Another embodiment is a composition comprising dDC and a polymer, wherein the composition is an aqueous liquid with a viscosity which increases upon contact with a surface of an eye, and wherein the viscosity of the composition is 12,000 cps or less, and wherein the viscosity of the composition becomes 70,000 or greater upon contact with the surface of the eye.

Another embodiment is a composition comprising dDC and a polymer, wherein the composition is an aqueous liquid with a viscosity which increases upon contact with a surface of an eye, and wherein the viscosity of said composition increases by a factor of 5 or more upon contact with the surface of the eye.

Another embodiment is a composition comprising dDC and a polymer, wherein the composition is an aqueous liquid with a viscosity which increases upon contact with a surface of an eye, and wherein the polymer is an anionic polymer.

Another embodiment is a composition comprising dDC and a polymer, wherein the composition is an aqueous liquid with a viscosity which increases upon contact with a surface of an eye, and wherein the polymer is an anionic polymer comprising a polymer of acrylic acid and a cellulose ether.

Another embodiment is a composition comprising dDC, a carbomer, and a cellulosic polymer.

Another embodiment is a composition comprising 0.8% dDC, 0.5% Methocel A4M premium, 0.3% Carbapol 934P, 2.2% glycerine, wherein the composition has a pH of 4.

Another embodiment is an aqueous composition comprising a therapeutically effective concentration of dDC, wherein the concentration of dDC is less than 1%.

Another embodiment is an aqueous composition comprising a therapeutically effective concentration of dDC, wherein the concentration of dDC is at least 0.4%.

Another embodiment is an aqueous composition comprising a therapeutically effective concentration of dDC, wherein the concentration of dDC is no greater than about 0.8%.

Another embodiment is an aqueous composition comprising a therapeutically effective concentration of dDC, wherein the concentration of dDC is about 0.8%.

Another embodiment is an eye drop comprising a therapeutically effective amount of dDC, wherein the amount of dDC is less than 300 μg.

Another embodiment is an eye drop comprising a therapeutically effective amount of dDC, wherein the amount of dDC is 280 μg or less.

Another embodiment is an eye drop comprising a therapeutically effective amount of dDC, wherein the amount of dDC is from 150 μg to 299 μg.

Another embodiment is a method comprising administering an effective amount of dDC topically to an eye of a person suffering from a viral infection, wherein less than 300 μL of dDC is administered to said eye.

Another embodiment is a method comprising administering an effective amount of dDC topically to an eye of a person suffering from a viral infection, wherein less than 300 μL of dDC is administered to said eye, wherein said viral infection is a disease selected from the group consisting of viral conjunctivitis, viral keratitis, viral trabeculitis, viral iritis, viral scleritis, viral blethritis, viral vitritis, viral uveitides, and combinations thereof.

Another embodiment is a method comprising administering an effective amount of dDC topically to an eye of a person suffering from a viral infection, wherein less than 300 μL of dDC is administered to said eye, wherein said viral infection is viral conjunctivitis or viral keratitis.

Another embodiment is a method comprising administering an effective amount of dDC topically to an eye of a person suffering from a viral infection, wherein less than 300 μL of dDC is administered to both eyes of the person no more than 3 times a day.

Another embodiment is a method comprising administering an effective amount of dDC topically to an eye of a person suffering from a viral infection, wherein less than 300 μl of dDC is administered to only one eye no more than 6 times a day.

Another embodiment is a kit comprising a composition and a dispenser, wherein said dispenser dispenses a drop comprising a therapeutically effective amount of dDC, wherein the amount of dDC is less than 300 μg.

Another embodiment is a composition comprising from 0% to 3% dDC and a penetration enhancer, wherein said composition is ophthalmically acceptable.

Reference to dDC herein should be broadly interpreted to mean dDC or a pharmaceutically acceptable salt thereof. A pharmaceutically acceptable salt is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

In the case that a concentration of dDC is disclosed, it should be taken to be the concentration of dDC in neutral form whether or not the dDC is present as a salt.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. A metabolite is broadly defined as a compound which is formed in vivo from the disclosed compound.

The amount of the presently useful compound or compounds administered is, of course, dependent on the therapeutic effect or effects desired, on the specific mammal being treated, on the severity and nature of the mammal's condition, on the manner of administration, on the potency and pharmacodynamics of the particular compound or compounds employed, and on the judgment of the prescribing physician.

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions of the present invention include, but are not limited to, benzalkonium chloride, chlorobutanol, thimerosal, phenylmercuric acetate and phenylmercuric nitrate. A useful surfactant is, for example, Tween 80. Likewise, various useful vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

The ingredients are usually used in the following amounts:

| Ingredient | Amount (% w/v) |
|---|---|
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound, and on the condition to be treated; the selection of the appropriate dose is well within the knowledge of the skilled artisan.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

### EXAMPLE 1

TABLE 1

| Ingredient | amount |
|---|---|
| dideoxycytidine (ddC) | 0.8% |
| Methocel A4M Premium | 0.5% |
| Carbapol 934P | 0.3% |
| Glycerin | 2.2% |
| pH | 4.0 |

The formulation shown in Table 1 was manufactured in one part. The Carbapol 934P was added to a 0.5% methocel solution. Glycerin was then added with mixing followed by dDC. The pH was then adjusted to 4.0 with 1 N sodium hydroxide or 1 N hydrochloric acid.

The composition of Table 1 had an approximate viscosity of 6000 to 12000 cps at pH 4.0, 25° C. and 70,000 to 120,000 cps at pH 7.0, 37° C.

### EXAMPLE 2

A naptholsulfonic acid salt of dDC was manufactured by adding dDC to a vial containing water. A stoichiometric amount of naphthol sulfonic acid potassium salt was solubilized in water in a second vial. The naphthol sulfonic acid solution was then added dropwise to the dDC solution and stirred for 30 minutes. The solution was evaporated to dryness over night yielding the naptholsulfonic acid salt of dDC.

The naptholsulfonic acid salt of dDC was formulated as a 1.65% w/v suspension. 330 mg of dideoxycytidine naphtholsulfonate was accurately weighed and transferred to a 20 mL vial. 20 mL of vehicle was to the vial. The suspension was the vigorously vortexed until homogeneous.

| Composition | % w/v |
|---|---|
| dideoxycytidine naphtholsulfonate | 1.65 |
| Sodium phosphate dibasic heptahydrate | 2.15 |
| Sodium phosphate monobasic monohydrate | 0.43 |
| Benzalkonium chloride | 0.005 |
| NaCL | 0.112 |
| Water | QS |
| NaOH, HCL | Adjust to pH 7 |

### EXAMPLE 3

### In Vivo Antiviral Efficacy Study

### Study Design

The antiviral efficacy of three (3) formulations of dDC [0.8% dDC (Example 1), 35 μL drop], 3.5% dDC (0.9% Saline Formulation, pH 5.5, 8 μL drop), 1.65% dDC (Example 2), 35 μL drop] against Ad5 were evaluated in 1 rabbit experiment using 25 rabbits. The experiment consisted of 5 groups of 5 rabbits. Of the 5 groups, 2 will be controls (0.5% cidofovir [positive antiviral control], 35 μL drop) and PBS [negative antiviral control, 35 μL drop] and 3 were the dDC formulations.

Ocular swabbing to recover adenovirus from the tear film and corneal and conjunctival surfaces were performed on days 0, 1, 3, 4, 5, 7, 9, 11, and 14 after inoculation at least 1 hr after the final dose of drug. Swabs were frozen at −70° C. pending viral assay.

### Outcome

The outcomes of the study are as follows:

### Positive Cultures Per Total

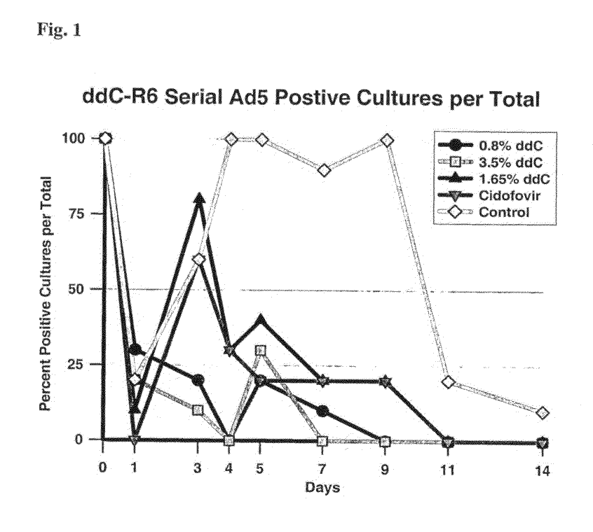

This outcome measure, shown in FIG. 1, is the most stringent measure of global adenovirus replication over the entire course of the experiment since one virus plaque constitutes a positive culture.

### Mean Duration of Shedding

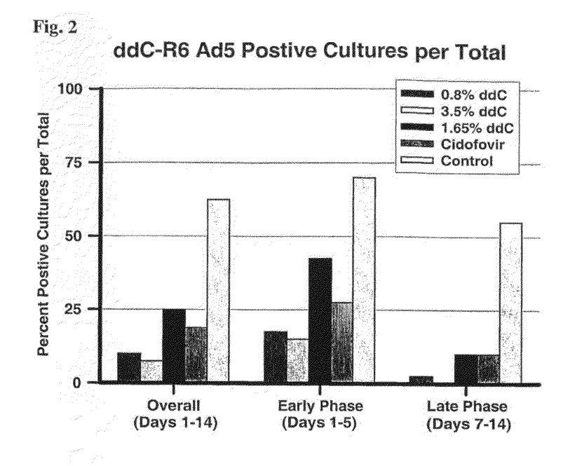

This outcome measure, shown in FIG. 2, determines the mean length of the infection.

### Mean Daily Ocular Titers

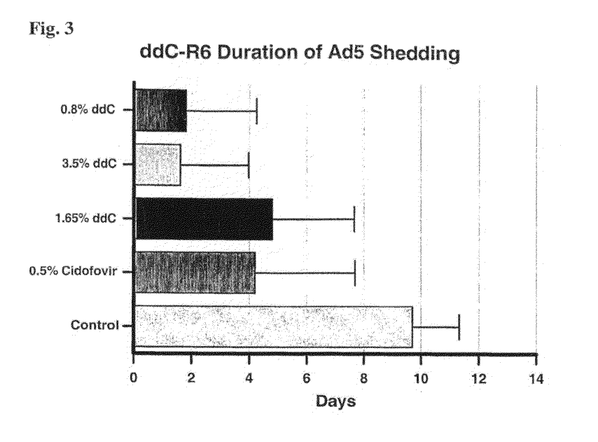

These outcome measures, shown in FIG. 3, determine the viral replication on each day of the experiment.

### EXAMPLE 4

The composition shown in Table 2 contains Brij 35 as an absorption enhancer:

TABLE 2

| Ingredient | amount |
|---|---|
| dideoxycytidine (ddC) | 0.8% |
| Sodium carboxymethylcellulose | 0.5% |
| Polyoxyethylene (23) lauryl ether (Brij 35) | 1.0 |
| Sodium Chloride | 0.50 |
| Potassium Chloride | 0.14 |
| Sodium Lactate | 0.30 |
| Calcium Chloride, Dihydrate | 0.02 |
| Magnesium Chloride, Hexahydrate | 0.006 |
| Benzalkonium Chloride | 0.002 |

The formulation is manufactured in two parts:

Part I is manufactured in the main batch vessel equipped with a propeller mixer. Purified water is charged to the tank and mixing is initiated. Sodium carboxymethylcellulose is then added and mixed until dispersed.

Part II is manufactured in a secondary vessel. Purified water is charged to the tank and mixing is initiated. Polyoxyethylene (23) lauryl ether (Brij 35), Sodium Chloride, Potassium Chloride, Sodium Lactate, Calcium Chloride, Magnesium Chloride and Benzalkonium Chloride are sequentially added allowing each to dissolve before adding the next. dDC is then added with mixing until dissolved

After the two aqueous phases are manufactured, (Part II) is transferred into the bulk phase (Part I) in the main batch vessel while mixing and thoroughly mixed for a designated time. Sodium Hydroxide or Hydrochloric acid is used to adjust the pH to within specifications.

The bulk product is then transferred to the filling area where the product is sterile filtered and filled into empty, low density polyethylene bottles.

### EXAMPLE 5

The composition shown in Table 3 contains a lipophilic salt of dDC, dDC-palmoate.

TABLE 3

| Ingredient | amount |
|---|---|
| Dideoxycytidine-palmoate | 1.0% w/w |
| Sodium carboxymethylcellulose | 2.0% w/w |
| Water | 97% w/w |

The formulation is manufactured by adding the weighed Dideoxycytidine-palmoate to a 2% aqueous solution of sodium carboxymethylcellulose. The suspension is then mixed until homogeneously dispersed.

What is claimed is:

1. Use of a compound in the manufacture of a medicament for the treatment of a viral infection affecting the eye, wherein said compound is dDC, and said medicament is an aqueous liquid with a polymer having a viscosity which increases upon contact with a surface of an eye.

2. Use of claim 1 wherein the viscosity of said medicament is pH dependent.

3. Use of claim 1 wherein the polymer is an anionic polymer.

4. Use of claim wherein the medicament comprises 0.8% dDC, 0.5% Methocel A4M premium, 0.3% Carbapol 934P, 2.2% glycerine, and has a pH of 4.

5. Use of a compound in the manufacture of a medicament for the treatment of a viral infection affecting the eye, wherein said medicament is an aqueous composition, and said compound is dDC having a concentration less than 1%.

6. Use of 12 wherein the concentration of dDC is at least 0.4%.

7. Use of claim 14 wherein the concentration of dDC is about 0.8%.

8. A kit comprising a composition and a dispenser, wherein said dispenser dispenses a drop comprising a therapeutically effective amount of dDC, wherein the amount of dDC is less than 300 μg.

9. A composition comprising from 0% to 3% dDC and a penetration enhancer, wherein said composition is ophthalmically acceptable.
